(19) United States
(12) Patent Application Publication
(10) Pub. No.: US 2017/0245886 A1
(43) Pub. Date: Aug. 31, 2017

(54) TUMOR STABILIZING APPARATUS FOR A MEDICAL PROCEDURE

(71) Applicants: Neil Jeffrey WITCOMB, Toronto (CA); Joshua Lee RICHMOND, Toronto (CA)

(72) Inventors: Neil Jeffrey WITCOMB, Toronto (CA); Joshua Lee RICHMOND, Toronto (CA)

(21) Appl. No.: 15/509,719

(22) PCT Filed: Dec. 3, 2014
(86) PCT No.: PCT/CA2014/051163

§ 371 Date: Mar. 8, 2017

(51) Int. Cl. A61B 17/34

(52) U.S. Cl. CPC A61B 17/3421; A61B 10/0233

(57) ABSTRACT

An apparatus is provided for use in a medical procedure for stabilizing a target during approach of the apparatus towards the target. The apparatus comprises a body having a proximal end and a distal end, the distal end having a cavity formed therein, and a advanceable tip housed within the cavity. The advanceable tip is advanceable to engage a surface of the target.

### TECHNICAL FIELD

The present disclosure is generally related to neurosurgical or medical procedures where access to a tumor is needed, and more specifically to a tumor stabilizing apparatus for a medical procedure.

### BACKGROUND

In an access port based medical procedure, during the cannulation step when navigating to a tumor a common occurrence is a tumor roll. This is caused by the obturator approaching the tumor at the wrong angle and pushing the tumor to the side as opposed to penetrating the tumor. This complicates the medical procedure because the surgeon must then look for the tumor, which has shifted position at the end of the positioned access port. This complication can be harmful to patients.

Therefore, there is a need for an improved way of approaching a tumor in an access port based medical procedure.

### SUMMARY

One aspect of the present disclosure provides an apparatus for use in a medical procedure for stabilizing a target during approach of the apparatus towards the target. The apparatus comprises a body having a proximal end and a distal end, the distal end having a cavity formed therein, and an advanceable tip housed within the cavity. The advanceable tip is advanceable to engage a surface of the target. The apparatus may be substantially cylindrical. The apparatus may be substantially cylindrical and have a pointed tip at the distal end and a handle portion at the proximal end, where the cavity is formed at an end of the pointed tip. The target may include biological tissue or a tumor.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the insertion of an access port into a human brain, for providing access to internal brain tissue during a medical procedure;

FIG. 2 shows an exemplary navigation system to support minimally invasive access port-based surgery;

FIG. 3 is a block diagram illustrating a control and processing system that may be used in the navigation system shown in FIG. 2;

FIG. 4A is a flow chart illustrating a method involved in a surgical procedure using the navigation system of FIG. 2;

FIG. 4B is a flow chart illustrating a method of registering a patient for a surgical procedure as outlined in FIG. 4A;

FIGS. 5A and 5B are diagrams illustrating tumor roll during insertion of a conventional access port and obturator into the human brain;

FIG. 6 is a diagram illustrating an obturator shown both inserted into an access port and in isolation;

FIG. 7A is a diagram illustrating insertion of the obturator and access port of FIG. 6 into a human brain;

FIG. 7B is a diagram illustrating tip advancement of the obturator of FIG. 6;

FIG. 7C is a diagram illustrating advancement of the access port of FIG. 6 subsequent to tip advancement;

FIG. 7D is a diagram illustrating removal of the obturator of FIG. 6 subsequent to access port advancement;

FIG. 7E is a diagram illustrating the resting position of the inserted access port of FIG. 6; and

FIG. 8 is a diagram illustrating an alternate embodiment of an apparatus having an advanceable tip.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about”, “approximately”, and “substantially” are meant to cover variations that may exist in the upper and lower limits of the ranges of values, such as variations in properties, parameters, and dimensions. In one non-limiting example, the terms “about”, “approximately”, and “substantially” mean plus or minus 10 percent or less.

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof. Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

The present disclosure is generally related to medical procedures, neurosurgery, and minimally invasive port-based surgery in specific.

In the example of a port-based surgery, a surgeon or robotic surgical system may perform a surgical procedure involving tumor resection in which the residual tumor remaining after is minimized, while also minimizing the trauma to the healthy white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial location of the patient as understood by the surgeon and the surgical system is as accurate as possible.

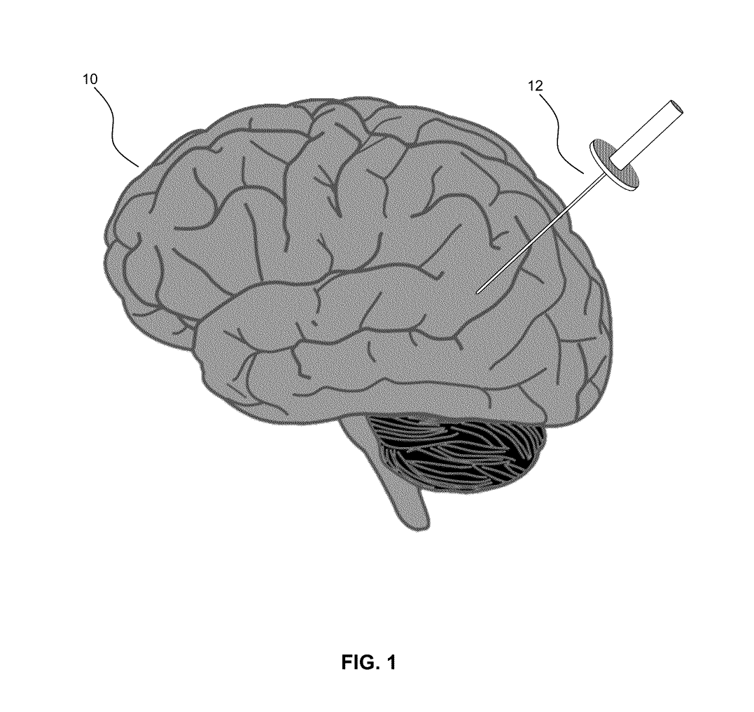

FIG. 1 illustrates the insertion of an access port into a human brain, for providing access to internal brain tissue during a medical procedure. In FIG. 1, access port 12 is inserted into a human brain 10, providing access to internal brain tissue. Access port 12 may include instruments such as catheters, surgical probes, or cylindrical ports such as the NICO Brain Path. Surgical tools and instruments may then be inserted within the lumen of the access port in order to perform surgical, diagnostic or therapeutic procedures, such as resecting tumors as necessary. The present disclosure applies equally well to catheters, DBS needles, a biopsy procedure, and also to biopsies and/or catheters in other medical procedures performed on other parts of the body where head immobilization is needed.

In the example of a port-based surgery, a straight or linear access port 12 is typically guided down a sulci path of the brain. Surgical instruments would then be inserted down the access port 12.

Optical tracking systems, which may be used in the medical procedure, track the position of a part of the instrument that is within line-of-site of the optical tracking camera. These optical tracking systems also require a reference to the patient to know where the instrument is relative to the target (e.g., a tumor) of the medical procedure. These optical tracking systems require a knowledge of the dimensions of the instrument being tracked so that, for example, the optical tracking system knows the position in space of a tip of a medical instrument relative to the tracking markers being tracked.

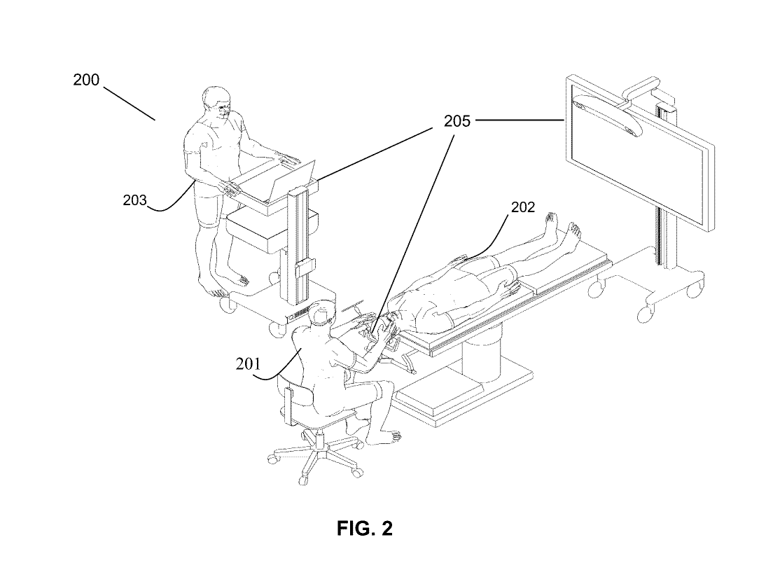

Referring to FIG. 2, an exemplary navigation system environment 200 is shown, which may be used to support navigated image-guided surgery. As shown in FIG. 2, surgeon 201 conducts a surgery on a patient 202 in an operating room (OR) environment. A medical navigation system 205 comprising an equipment tower, tracking system, displays and tracked instruments assist the surgeon 201 during his procedure. An operator 203 is also present to operate, control and provide assistance for the medical navigation system 205.

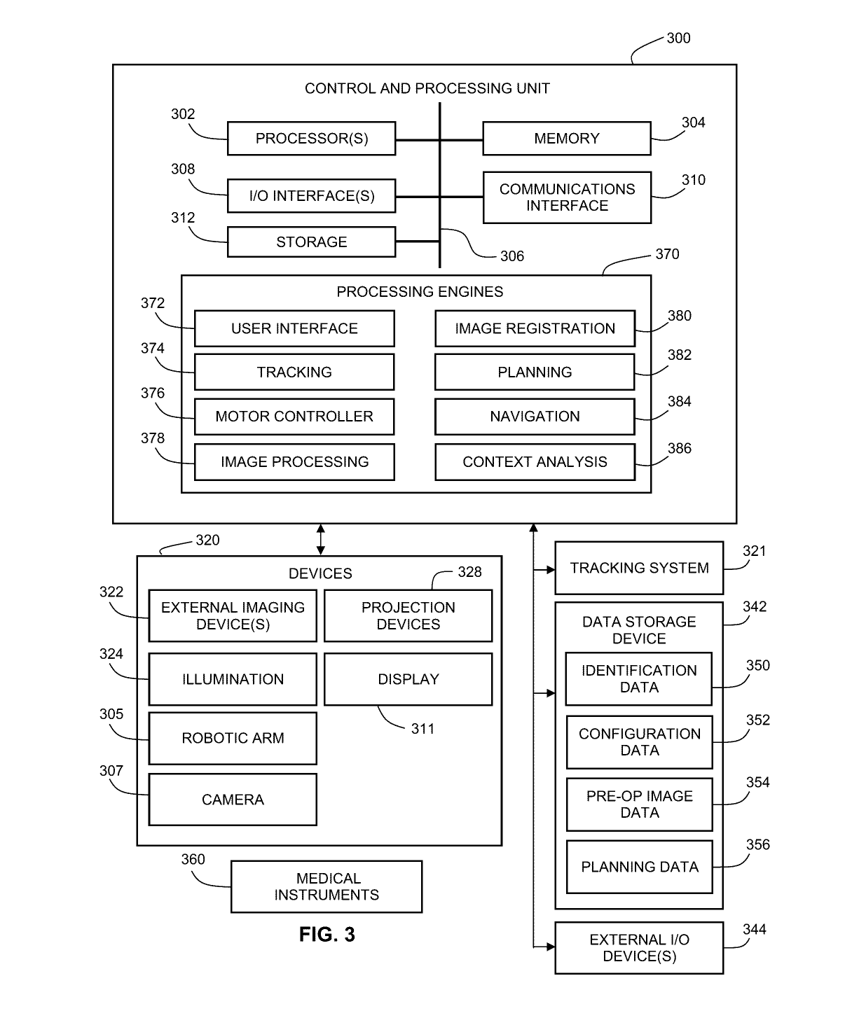

Referring to FIG. 3, a block diagram is shown illustrating a control and processing system 300 that may be used in the medical navigation system 200 shown in FIG. 3 (e.g., as part of the equipment tower). As shown in FIG. 3, in one example, control and processing system 300 may include one or more processors 302, a memory 304, a system bus 306, one or more input/output interfaces 308, a communications interface 310, and storage device 312. Control and processing system 300 may be interfaced with other external devices, such as tracking system 321, data storage 342, and external user input and output devices 344, which may include, for example, one or more of a display, keyboard, mouse, sensors attached to medical equipment, foot pedal, and microphone and speaker. Data storage 342 may be any suitable data storage device, such as a local or remote computing device (e.g. a computer, hard drive, digital media device, or server) having a database stored thereon. In the example shown in FIG. 3, data storage device 342 includes identification data 350 for identifying one or more medical instruments 360 and configuration data 352 that associates customized configuration parameters with one or more medical instruments 360. Data storage device 342 may also include preoperative image data 354 and/or medical procedure planning data 356. Although data storage device 342 is shown as a single device in FIG. 3, it will be understood that in other embodiments, data storage device 342 may be provided as multiple storage devices.

Medical instruments 360 are identifiable by control and processing unit 300. Medical instruments 360 may be connected to and controlled by control and processing unit 300, or medical instruments 360 may be operated or otherwise employed independent of control and processing unit 300. Tracking system 321 may be employed to track one or more of medical instruments 360 and spatially register the one or more tracked medical instruments to an intraoperative reference frame. For example, medical instruments 360 may include tracking markers such as tracking spheres that may be recognizable by a tracking camera 307. In one example, the tracking camera 307 may be an infrared (IR) tracking camera. In another example, as sheath placed over a medical instrument 360 may be connected to and controlled by control and processing unit 300.

Control and processing unit 300 may also interface with a number of configurable devices, and may intraoperatively reconfigure one or more of such devices based on configuration parameters obtained from configuration data 352. Examples of devices 320, as shown in FIG. 3, include one or more external imaging devices 322, one or more illumination devices 324, a robotic arm 305, one or more projection devices 328, and one or more displays 311.

Exemplary aspects of the disclosure can be implemented via processor(s) 302 and/or memory 304. For example, the functionalities described herein can be partially implemented via hardware logic in processor 302 and partially using the instructions stored in memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately in FIG. 3, in one example the processing modules 370 may be stored in the memory 304 and the processing modules may be collectively referred to as processing modules 370.

It is to be understood that the system is not intended to be limited to the components shown in FIG. 3. One or more components of the control and processing system 300 may be provided as an external component or device. In one example, navigation module 384 may be provided as an external navigation system that is integrated with control and processing system 300.

Some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

According to one aspect of the present application, one purpose of the navigation system 205, which may include control and processing unit 300, is to provide tools to the neurosurgeon that will lead to the most informed, least damaging neurosurgical operations. In addition to removal of brain tumors and intracranial hemorrhages (ICH), the navigation system 205 can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

While one example of a navigation system 205 is provided that may be used with aspects of the present application, any suitable navigation system may be used, such as a navigation system using optical tracking instead of infrared cameras.

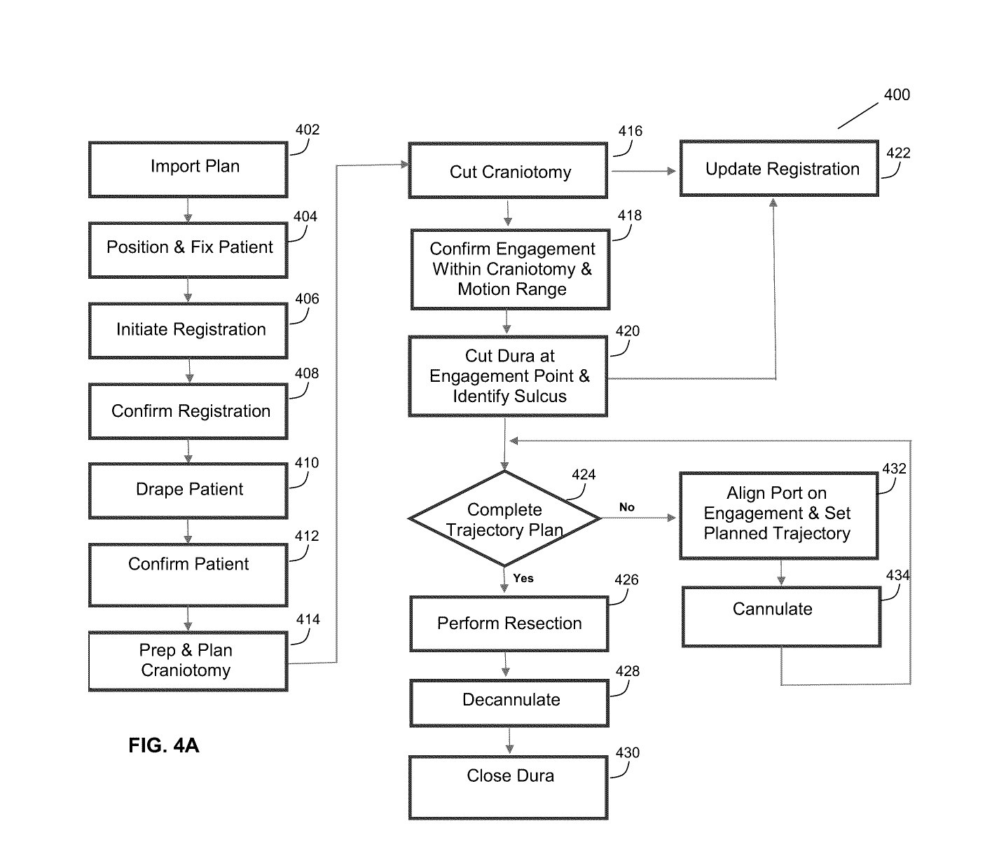

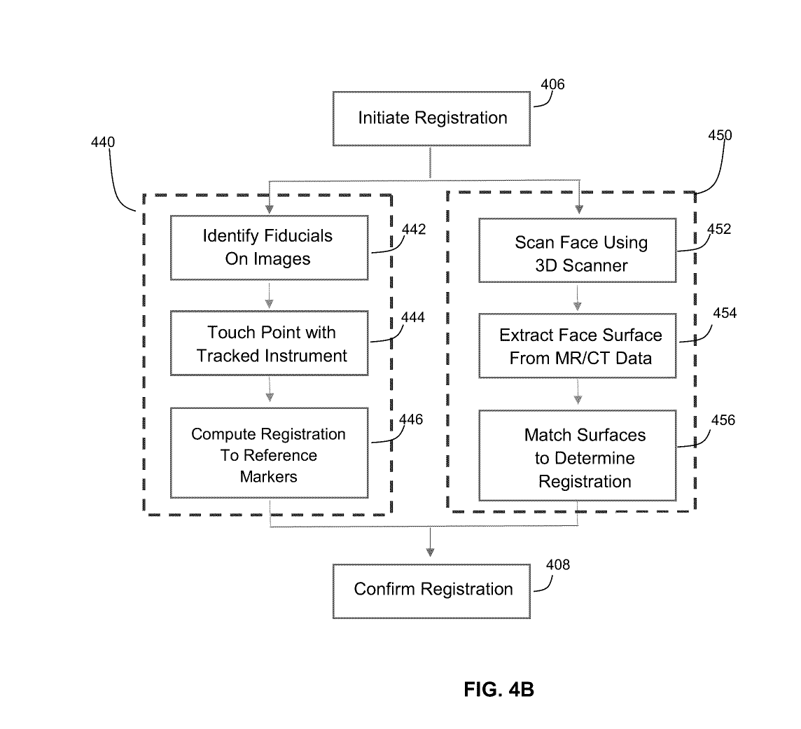

Referring to FIG. 4A, a flow chart is shown illustrating a method 400 of performing a port-based surgical procedure using a navigation system, such as the medical navigation system 205 described in relation to FIG. 2. At a first block 402, the port-based surgical plan is imported. A detailed description of the process to create and select a surgical plan is outlined in international publication WO/2014/139024, entitled “PLANNING, NAVIGATION AND SIMULATION SYSTEMS AND METHODS FOR MINIMALLY INVASIVE THERAPY”, which claims priority to U.S. Provisional Patent Application Ser. Nos. 61/800,155 and 61/924,993, which are all hereby incorporated by reference in their entirety.

Once the plan has been imported into the navigation system at the block 402, the patient is placed on a surgical bed. The head position is confirmed with the patient plan in the navigation system (block 404), which in one example may be implemented by the computer or controller forming part of the equipment tower 201.

Next, registration of the patient is initiated (block 406). The phrase “registration” or “image registration” refers to the process of transforming different sets of data into one coordinate system. Data may include multiple photographs, data from different sensors, times, depths, or viewpoints. The process of “registration” is used in the present application for medical imaging in which images from different imaging modalities are co-registered. Registration is used in order to be able to compare or integrate the data obtained from these different modalities to the patient in physical space.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT to patient in physical space.

Referring now to FIG. 4B, a flow chart is shown illustrating a method involved in registration block 406 as outlined in FIG. 4A, in greater detail. If the use of fiducial touch points (440) is contemplated, the method involves first identifying fiducials on images (block 442), then touching the touch points with a tracked instrument (block 444). Next, the navigation system computes the registration to reference markers (block 446).

Alternately, registration can also be completed by conducting a surface scan procedure (block 450). The block 450 is presented to show an alternative approach, but may not typically be used when using a fiducial pointer. First, the face is scanned using a 3D scanner (block 452). Next, the face surface is extracted from MR/CT data (block 454). Finally, surfaces are matched to determine registration data points (block 456).

Upon completion of either the fiducial touch points (440) or surface scan (450) procedures, the data extracted is computed and used to confirm registration at block 408, shown in FIG. 4A.

Referring back to FIG. 4A, once registration is confirmed (block 408), the patient is draped (block 410). Sometimes block 410 and block 408 are performed in the opposite order because in some situations the draping my cause the registration to become invalid. Typically, draping involves covering the patient and surrounding areas with a sterile barrier to create and maintain a sterile field during the surgical procedure. The purpose of draping is to eliminate the passage of microorganisms (e.g., bacteria) between non-sterile and sterile areas. At this point, conventional navigation systems require that the non-sterile patient reference is replaced with a sterile patient reference of identical geometry location and orientation. Numerous mechanical methods may be used to minimize the displacement of the new sterile patient reference relative to the non-sterile one that was used for registration but it is inevitable that some error will exist. This error directly translates into registration error between the surgical field and pre-surgical images. In fact, the further away points of interest are from the patient reference, the worse the error will be.

Upon completion of draping (block 410), the patient engagement points are confirmed (block 412) and then the craniotomy is prepared and planned (block 414).

Upon completion of the preparation and planning of the craniotomy (block 414), the craniotomy is cut and a bone flap is temporarily removed from the skull to access the brain (block 416). Registration data is updated with the navigation system at this point (block 422).

Next, the engagement within craniotomy and the motion range are confirmed (block 418). Next, the procedure advances to cutting the dura at the engagement points and identifying the sulcus (block 420).

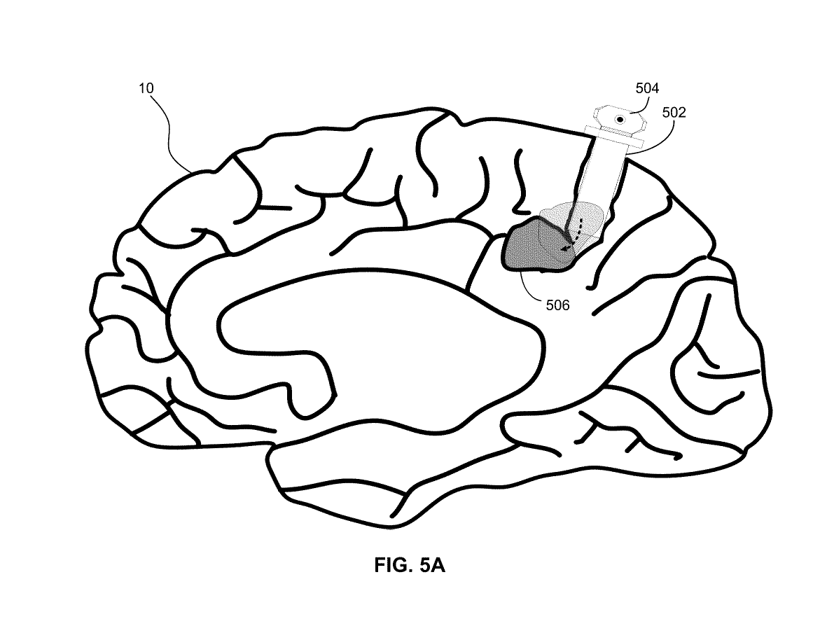

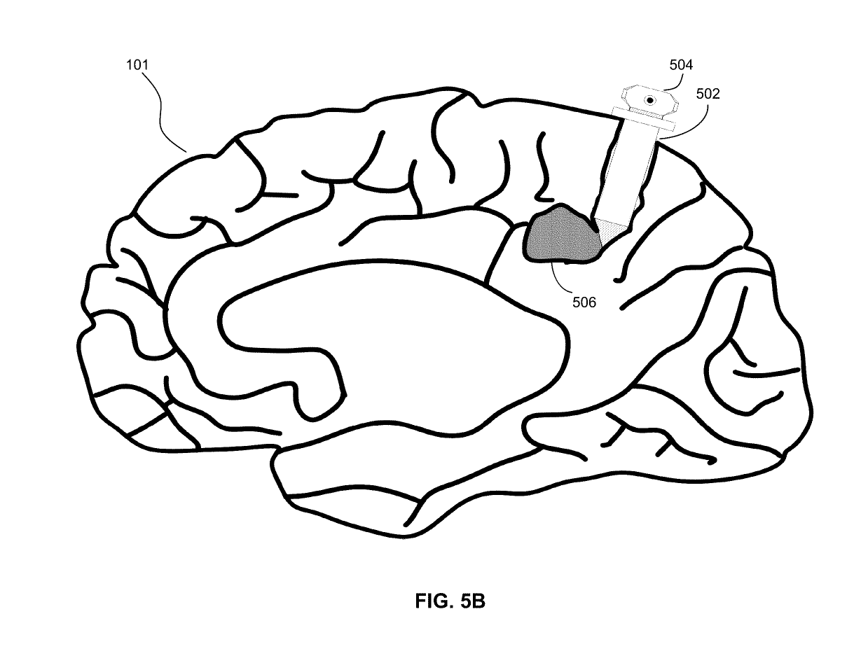

Thereafter, the cannulation process is initiated (block 424). Cannulation involves inserting a port into the brain, typically along a sulci path as identified at 420, along a trajectory plan. Cannulation is typically an iterative process that involves repeating the steps of aligning the port on engagement and setting the planned trajectory (block 432) and then cannulating to the target depth (block 434) until the complete trajectory plan is executed (block 424). The cannulation process is described in more detail below in connection with FIGS. 5-7.

Once cannulation is complete, the surgeon then performs resection (block 426) to remove part of the brain and/or tumor of interest. The surgeon then decannulates (block 428) by removing the port and any tracking instruments from the brain. Finally, the surgeon closes the dura and completes the craniotomy (block 430). Some aspects of FIG. 4A are specific to port-based surgery, such as portions of blocks 428, 420, and 434, but the appropriate portions of these blocks may be skipped or suitably modified when performing non-port based surgery.

Referring to FIGS. 5A and 5B, diagrams are shown illustrating a tumor roll during insertion of a conventional access port 502 and obturator 504 into the human brain. As shown in FIG. 5A, the conventional access port 502 and obturator 504 are inserted into the human brain 10. According to conventional practice with such a conventional access port 502 and obturator 504, the access port 502 and obturator 504 are advanced until the access port 502 is in the desired position for the medical procedure, which means that the access port 502 and/or the obturator 504 will contact tumor 506. When this is performed during a surface cannulation, the lesion or tumor 506 may roll away because the tip of the obturator 504 pushes against the tumor 506, depending on how the tumor 506 was approached. FIG. 5A shows the tumor 506 being pushed away by obturator 504 and FIG. 5B shows the tumor 506 in its new resting position once cannulation is complete. The movement of the tumor 506 means that the surgeon must then look for the tumor 506 once the access port 502 is in place and the obturator 504 is removed, which is contradictory to the intended purpose of the access port 502, which is intended to provide the surgeon with a direct line of access (and line of sight) to the tumor 506.

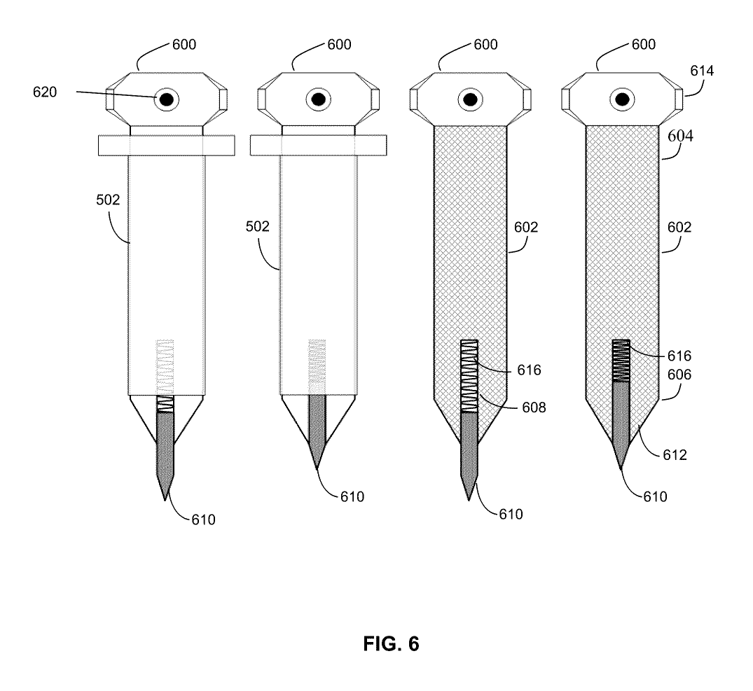

Referring to FIG. 6, a series of four diagrams are shown illustrating an apparatus 600 shown both inserted into an access port 502 and in isolation. The apparatus 600 is shown as a sectional view such that the interior of apparatus 600 is shown in FIG. 6. In one example, the apparatus 600 may be an obturator, however aspects of the present description may be applied to a variety of medical devices or tools. The apparatus 600 may be for use in a medical procedure for stabilizing a tumor, such as the tumor 506, during approach of the apparatus 600 towards the tumor 506. The apparatus 600 includes a body 602 having a proximal end 604 and a distal end 606. The distal end 606 has a cavity 608 formed therein. A advanceable tip 610 is housed within the cavity 608. The advanceable tip 610 may be advanceable to engage a surface of the tumor.

In one example, the apparatus 600 may be substantially cylindrical. However, apparatus 600 may be constructed in any suitable shape and form to meet the design criteria of a particular application. In another example, the apparatus 600 may be substantially cylindrical and may have a pointed tip 612 at the distal end 606 and a handle portion 614 at the proximal end 604. The cavity 608 may be formed within distal end 606 and emerge from the apparatus 600 at an end of the pointed tip 612. The handle portion 614 may also contain screw hole 620 where a screw may be placed to secure the apparatus 600 in place during operation. Screw hole 620 may also receive a tracking probe to enable apparatus 600 to be tracked in a navigation system.

In one example, the advanceable tip 610 may be spring loaded with a spring 616 and is advanceable into a tumor upon release of the spring. In other words, when the apparatus 600 is advanced into the human brain 10 and a tip of the advanceable tip 610 is resting in a retracted position close to the tumor 506, the spring may be released (e.g., with a button or control located on handle 614) causing the advanceable tip 610 to advance and penetrate tumor 506 at least partially. In another example, the advanceable tip 610 may be connected to an electric actuator and is advanceable into a tumor upon activation of the actuator (e.g., with a button or control located on handle 614 or elsewhere). In another example, the advanceable tip 610 may be pneumatically controlled and is advanceable into a tumor upon activation of the pneumatic control (e.g., with a button or control located on handle 614 or elsewhere). While examples of springs, electric motors, and air pressure are provided as possible actuating mechanisms for advancing and retracting the advanceable tip 610, any suitable actuating means may be used to meet the design criteria of a particular application. The advanceable tip 610 may penetrate the tumor 506 at least partially upon advancement therefore stabilizing the tumor 506 and preventing tumor 506 movement during a subsequent stage of the medical procedure (e.g., when the access port 502 is advanced into position over the obturator 600). In one example, the advanceable tip 610 may penetrate the tumor 506 upon advancement due to high deployment speed of the tip 610.

In one example, the subsequent stage of the medical procedure, referred to above, may include advancement of the access port 502, where the access port 502 advances by sliding on an outside surface of the apparatus 600 to a desired position, after which the apparatus 600 is removed from the access port 502 when the access port 502 is in its desired position inside the brain 10. In another example, the subsequent stage of the medical procedure may be taking a biopsy sample of the brain 10.

In one example, the apparatus 600 may be an obturator for facilitating placement of the access port 502. In another example, the apparatus 600 may be a biopsy probe having an advanceable tip similar to the advanceable tip 610. The advanceable tip 610 may take a number of forms including a corkscrew tip, a biopsy needle tip, a pointed needle tip (e.g., such as that shown in FIG. 6), or an alligator clip tip. While some examples of a suitable tip 610 are provided, the advanceable tip 610 may take any suitable form to meet the design criteria of a particular application.

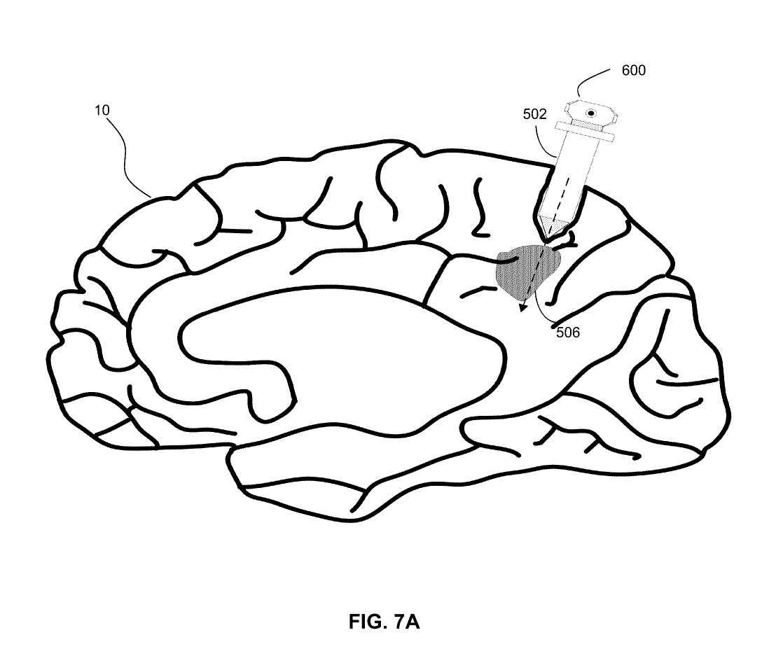

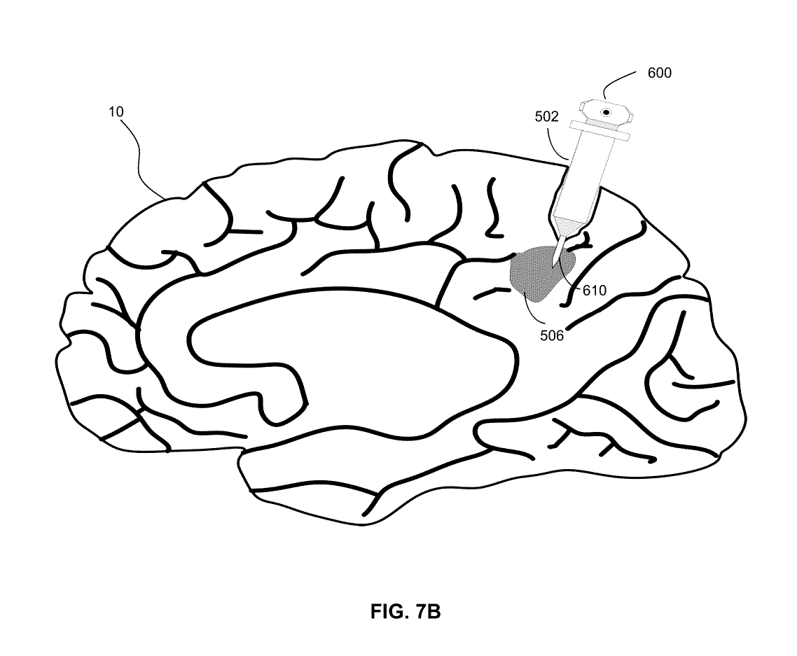

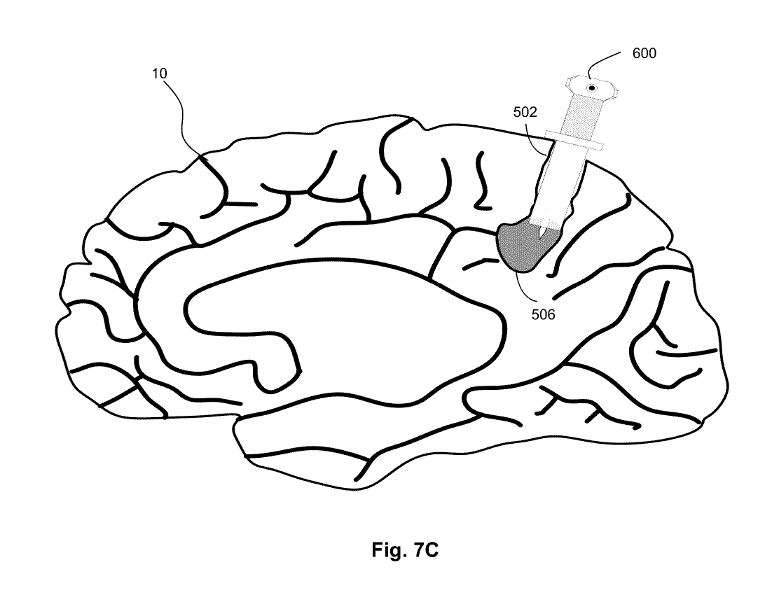

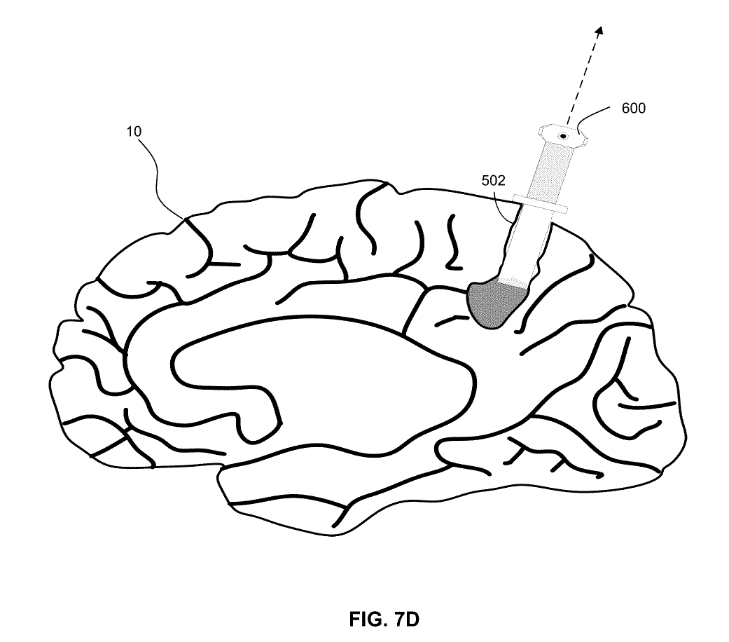

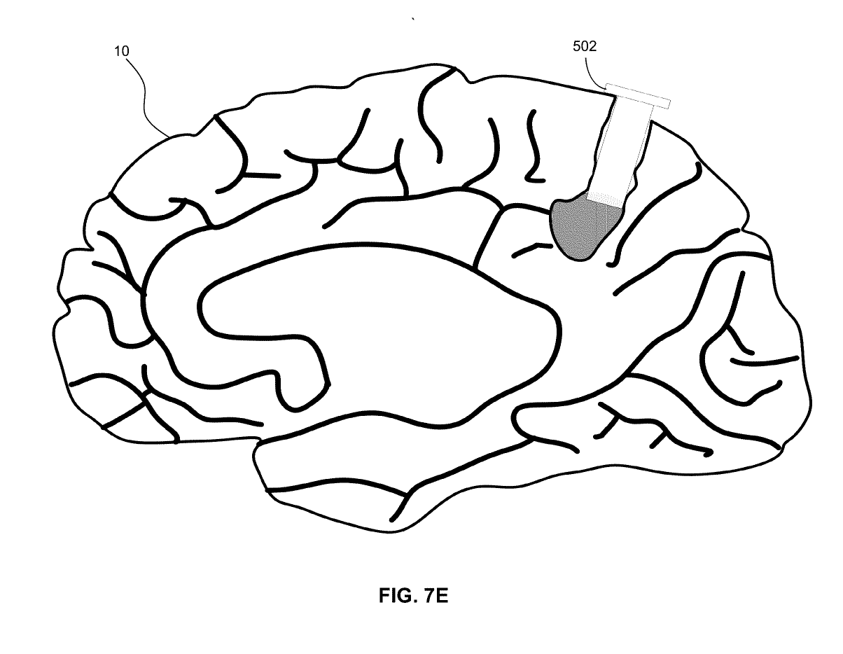

Referring now to FIG. 7A, a diagram is shown illustrating insertion of the apparatus 600 and access port 502 of FIG. 6 into the human brain 10. FIG. 7B shows a diagram illustrating tip deployment of the apparatus 600 of FIG. 6. FIG. 7C is a diagram illustrating advancement of the access port 502 of FIG. 6 subsequent to obturator tip deployment. FIG. 7D is a diagram illustrating removal of the apparatus 600 of FIG. 6 subsequent to access port 502 advancement. FIG. 7E is a diagram illustrating the inserted resting position of the access port 502 of FIG. 6. FIGS. 7A-7E will now be discussed concurrently. The example depicted in FIGS. 7A-7E provides the example where the apparatus 600 is an obturator. As such, the apparatus 600 will be referred to below as the obturator 600. However, it should be understood based on the description provided above that the present application is equally applicable to other medical devices such as biopsy probes, etc.

In order to place an access port 502 into position inside the brain 10, an obturator 600 is first placed inside the access port 502. In this regard the obturator 600 may have an outside diameter that is approximately equal to or slightly less than an inside diameter of the access port 502. The obturator 600 is then used to guide the access port 502 into position inside the brain 10, as shown in FIG. 7A. As shown in FIG. 7A, the first step of the process stops when the advanceable tip 610 closely approaches the tumor 506.

As shown in FIG. 7B, when the advanceable tip 610 closely approaches the tumor 506, the advanceable tip 610 is deployed, at least partially penetrating the tumor 506. With the advanceable tip 506 at least partially lodged in the tumor 506, the tumor 506 is then significantly prevented from moving or rolling out of its original position.

As shown in FIG. 7C, with the advanceable tip 610 at least partially lodged in the tumor 506, the access port 502 may then be slid into the desired position with a distal end of the access port 502 positioned directly adjacent or even contacting tumor 506 without the risk of the tumor 506 substantially moving or rolling out of position.

As shown in FIG. 7D, once the access port 502 is in the desired position, obturator 600 may then be removed from the access port 502, leaving the access port 502 in its final desired resting position, as shown in FIG. 7E. In the example where obturator 600 has a spring 616, advanceable tip 610 may be manually pushed back into obturator 600 after removal and/or cleaning and/or sterilization. In the example where obturator 600 has an electrically or pneumatically controlled advanceable tip 610, the advanceable tip 610 may be retracted prior to removing the obturator 600 from access port 502, for example by pushing the same or a different button or control on the handle 614.

On aspect of the present disclosure provides a mechanical or electromechanical system designed to stabilize a hard (e.g., non-cystic) tumor, such as during a brain tumor resection surgery. The system may be integrated with or used with an obturator as discussed and shown above. During the cannulation step of a surgery when navigating to the tumor, a common occurrence is a tumor roll. This is caused by the obturator approaching the tumor at the wrong angle and pushing it to the side as opposed to penetrating the tumor, as previously discussed. As aspect of the present description integrates a harpoon type higher velocity penetration device to anchor the tumor before the obturator penetrates the tumor, such as a needle or a biopsy needle. Once the obturator reaches the vicinity of the tumor, the obturator may fire the stabilizing needle to anchor the tumor in place. The needle may penetrate the tumor and once the tumor is stabilized the port may be cannulated to its final position and the obturator removed.

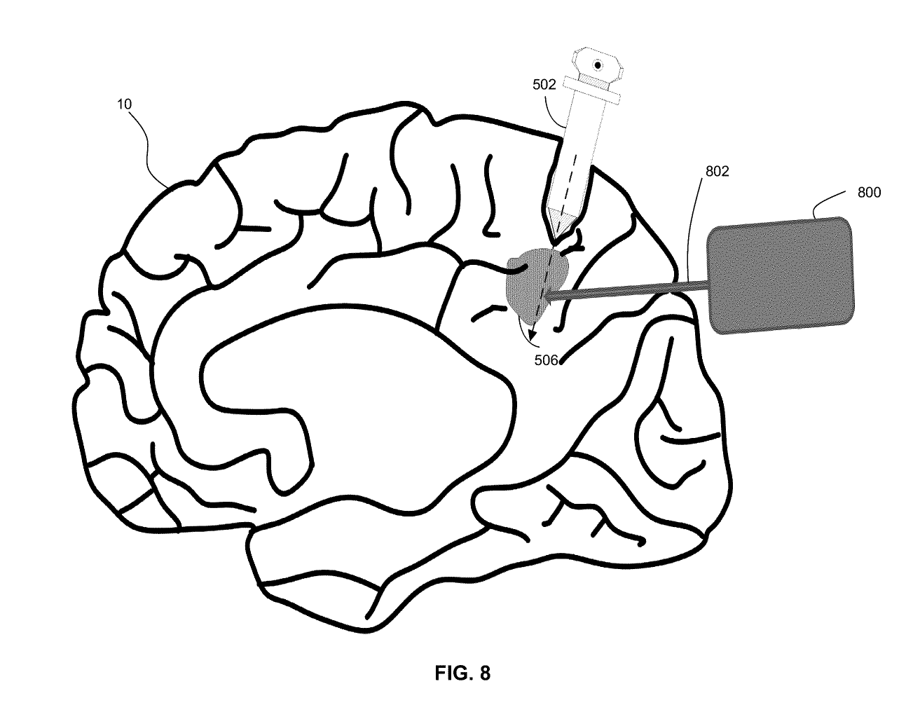

Referring now to FIG. 8, a diagram is shown illustrating an alternate embodiment of an apparatus 800. In one example, the apparatus 800 may be a pointed or barbed tool having a narrow shaft 802 that is used to skewer the target, such as tumor 506, via a small burr hole that may be formed in the skull and/or brain. The apparatus 800 may hold the target in place during approach of the access port 502. The apparatus 800 may also have an advanceable tip, similar to any of the advanceable tips 610 discussed above.

The present application may be applicable in areas of the body where a tumor roll may occur, such as in soft tissue. For example, in the prostate the tissue is typically stable, but not always. Dense tumors may need to be anchored. Cystic tumors for example may typically not be anchored but also will typically not roll. The obturator will generally stop short of the tumor before the needle is fired to assure the tumor doesn't partially shift position.

The specific embodiments described above have been shown by way of example, and it should be understood that these embodiments may be susceptible to various modifications and alternative forms. It should be further understood that the claims are not intended to be limited to the particular forms disclosed, but rather to cover modifications, equivalents, and alternatives falling within the spirit and scope of this disclosure.

We claim:

1. An apparatus for use in a medical procedure for stabilizing a target during approach of the apparatus towards the target, the apparatus comprising:
a body having a proximal end and a distal end, the distal end having a cavity formed therein; and
an advanceable tip housed within the cavity in a resting position, the advanceable tip being advanceable to engage a surface of the target in an engaged position.

2. The apparatus according to claim 1, wherein the apparatus is substantially cylindrical.

3. The apparatus according to claim 1, wherein the apparatus is substantially cylindrical and has a pointed tip at the distal end and a handle portion at the proximal end, the cavity formed at an end of the pointed tip.

4. The apparatus according to any one of claims 1-3, wherein the apparatus includes an obturator for facilitating placement of an access port.

5. The apparatus according to any one of claims 1-3, wherein the apparatus includes a biopsy probe.

6. The apparatus according to any one of claims 1-5, wherein the advanceable tip includes any one of a corkscrew tip, a biopsy needle tip, a pointed needle tip, and an alligator clip tip.

7. The apparatus according to any one of claims 1-6, wherein the advanceable tip is spring loaded and is advanceable into the target on release of the spring.

8. The apparatus according to any one of claims 1-6, wherein the advanceable tip is connected to an electric actuator and is advanceable into the target on activation of the actuator.

9. The apparatus according to any one of claims 1-6, wherein the advanceable tip is pneumatically controlled and is advanceable into the target on activation of the pneumatic control.

10. The apparatus according to any one of claims 1-9, wherein the advanceable tip impales the target on advancement therefore stabilizing the target and preventing target movement during a subsequent stage of the medical procedure.

11. The apparatus according to claim 10, wherein the advanceable tip penetrates the target upon advancement due to high deployment speed of the tip.

12. The apparatus according to claim 10, wherein the subsequent stage of the medical procedure includes advancement of an access port, where the access port advances by sliding on an outside surface of the apparatus to a desired position, after which the apparatus is removed from the access port.

13. The apparatus according to claim 10, wherein the subsequent stage of the medical procedure includes taking a biopsy.

14. The apparatus according to any one of claims 1-13, wherein the apparatus further includes an obturator.

15. The apparatus according to any one of claims 1-14, wherein the target includes a tumor.

16. The apparatus according to any one of claims 1-15, wherein the target includes biological tissue.

17. The apparatus according to any one of claims 1-16, wherein the advanceable tip is advanced by actuation of a control located on the proximal end of the body.

18. The apparatus according to claim 17, wherein the control includes a button.
